(19) United States
(12) Patent Application Publication
(10) Pub. No.: US 2021/0137449 A1
(43) Pub. Date: May 13, 2021

(54) DIAGNOSING SYSTEM AND METHOD USING VOICE DATA

(71) Applicant: OLYMPUS CORPORATION, Tokyo (JP)

(72) Inventors: Hirokazu NOZAKI, Hachioji-shi (JP); Wataru HASHIMOTO, Kawasaki-shi (JP); Takashi SUZUKI, Hino-shi (JP); Mitsunori KUBO, Hachioji-shi (JP); Yusuke KUWA, Hachioji-shi (JP); Yuji SAKAMOTO, Kunitachi-shi (JP); Mariko OTAGIRI, Sagamihara-shi (JP); Naoki YOSHIDA, Mitaka-shi (JP); Yoji WATANABE, Fuchu-shi (JP)

(73) Assignee: OLYMPUS CORPORATION, Tokyo (JP)

(21) Appl. No.: 16/681,276

(22) Filed: Nov. 12, 2019

(51) Int. Cl. A61B 5/00; G10L 15/22; G10L 25/66; G10L 25/18

(52) U.S. Cl. CPC A61B 5/4803; G10L 15/22; A61B 5/4227; G10L 25/18; A61B 5/4233; G10L 25/66

(57) ABSTRACT

A system that can include a processor that obtains voice data of a subject. The processor can compare the voice data of the subject with reference normal and abnormal voice data. If the voice data of the subject is abnormal, the processor can generate an abnormal subject voice data that is indicative of differences between the subject voice data and the normal voice data. By comparing the generated abnormal subject voice data with the reference normal and abnormal voice data, the processor can determine what condition the subject may be suffering from and the severity of that condition.

### BACKGROUND

There are different medical conditions and diseases that cause distortion in a person's voice. For example, infectious diseases such as the common cold or influenza cause inflammation of the throat which results in voice distortion. Growths on the vocal cords such as nodules, polyps, and cysts can cause hoarseness in the voice. Many diseases result in growths on the vocal cords.

For example, thyroid cancer can cause nodules to grow on the thyroid. Because the thyroid gland is in close proximity to the larynx or voice box, the thyroid nodule may press on the voice box to cause changes in a person's voice. Esophageal cancer can also cause hoarseness in the voice. Early detection in changes in a person's voice can be helpful for early detection and treatment of these cancers.

Throat cancer can also result in changes in the voice, such as hoarseness or not speaking clearly. Throat cancers grow very quickly, and thus, early detection is very desirable for early treatment and increased chance of survival.

Conventionally, diagnosis of the above-mentioned cancers and other diseases that cause growths on the vocal cords involve using an endoscopic voice data, computerized tomography (CT), magnetic resonance imaging (MRI) or X-ray voice data. Audio voice data may be used as a reference but typically this is newly acquired voice data which may already be different from a patient's normal voice. Diagnoses are typically performed by highly trained medical experts or technicians. Determining the degree of severity of the diagnosed condition typically can only be made by a highly trained medical expert.

### SUMMARY

The present systems and methods provide quantitative and objective diagnostic support information from voice data to facilitate consistent and accurate diagnoses of conditions that affect the throat or vocal cords, such as thyroid, throat and esophageal cancer, infectious diseases and growths on vocal cords.

For example, the system can include a processor that obtains voice data of a subject. The processor can compare the voice data of the subject with reference normal and abnormal voice data. If the voice data of the subject is abnormal, the processor can generate an abnormal subject voice data that is indicative of differences between the subject voice data and the normal voice data. By comparing the generated abnormal subject voice data with the reference normal and abnormal voice data, the processor can determine what condition the subject may be suffering from and the severity of that condition.

The method may be similarly implemented as described above.

### BRIEF DESCRIPTION OF THE DRAWINGS

FIG. 1 is a block diagram of an exemplary diagnostic processor system.

FIG. 2 is a flow chart of an exemplary diagnosis method.

FIG. 3 is a flow chart of an exemplary method for generating voice data of a subject.

FIG. 4 is a flow chart of an exemplary method for categorizing reference and standard data.

FIG. 5 is a flow chart of an exemplary method for generating and analyzing abnormal data.

FIG. 6 is a flow chart of an exemplary method for diagnosing a subject based on a comparison of a subject's abnormal voice data and reference abnormal voice data.

FIG. 7 is a flow chart of an exemplary method for generating abnormal scores for a subject.

FIG. 8 is a flow chart of an exemplary method for generating non-voice abnormality scores for a subject.

FIG. 9 is a table of an exemplary method for determining the severity of a subject's condition based on his calculated score.

FIG. 10 is a graph of an exemplary method for calculating a subject's score.

FIG. 11 is a flow chart of an exemplary method for determining a severity of a subject's condition.

FIGS. 12A-C shows an exemplary method for determining vocal characteristics such as spectral envelope data.

FIG. 13 shows spectral envelope data being divided into a plurality of regions.

### DETAILED DESCRIPTION OF EMBODIMENTS

It will be apparent to the skilled artisan in the medical field from this disclosure that the following descriptions of exemplary embodiments are provided as examples only and need not limit the broad inventive principles described herein or included in the appended claims.

The present disclosure relates to a diagnostic system, computer-readable storage medium, and method for determining a condition of a subject based on audio voice data. In an exemplary embodiment, audio voice data relating to the voice of a subject is compared with audio voice data sets belonging to large populations of people stored on a server. It can then be determined whether the voice data is abnormal voice data. The subject's abnormal voice data is then compared with reference abnormal voice data representative of different severity levels of conditions that contribute to an abnormal or altered voice. The subject is then diagnosed based at least in part on the comparison between the subject's abnormal voice data and the reference abnormal voice data as having a particular condition. As discussed in more detail below, the system and the associated computer-readable storage medium and method enable consistent and accurate diagnoses of different conditions that contribute to an abnormal or altered voice and different severity levels.

The diagnostic system, computer readable medium and method can be implemented on a mobile device application. The mobile device can record and store a normal voice of a subject. The application can then monitor and detect whether the subject's voice is abnormal. When an abnormal voice is detected, the mobile device can provide the user with a warning which can be an audio or visual warning.

FIG. 1 shows an exemplary processor system 10 for use in connection with diagnosing diseases that affect the voice. The processor system 10 may be a general-purpose computer, such as a personal computer, a specific-purpose computer or workstation, a mainframe computer, or a distributed computing system. The processor system 10 is configured to execute various software programs, including software performing all or part of the processes and algorithms disclosed herein. The exemplary processor system 10 includes a controller or processor 12 that is configured to process data, such voice data and non-voice data information received as inputs for various algorithms and software programs. The processor 12 may include hardware, and the hardware may include at least one of a circuit for processing digital signals and a circuit for processing analog signals, for example. The processor may include one or a plurality of circuit devices (e.g., an IC) or one or a plurality of circuit elements (e.g., a resistor, a capacitor) on a circuit board, for example. The processor 12 may be a central processing unit (CPU), and/or various types of processors, including a GPU (Graphics Processing Unit) and a DSP (Digital Signal Processor), may be used. The processor may be a hardware circuit with an ASIC (Application Specific Integrated Circuit) or an FPGA (Field-Programmable Gate Array). The processor may include an amplification circuit, a filter circuit, or the like for processing analog signals. The processor system 10 can include a microcontroller or a microprocessor, such as a central processing unit (CPU) that executes various functions for the system 10.

The processor 12 may execute operating system instructions, along with software algorithms, computer-executable instructions, and processing functions of the system 10. Such algorithms and computer-executable instructions may be stored in a computer readable-storage medium, such as storage 14. “Computer readable-storage medium” as used herein refers to a non-transitory computer readable storage medium. The storage 14 may include a memory and/or other storage device. The memory may be, for example, random-access memory (RAM) of a computer. The memory may be a semiconductor memory such as an SRAM and a DRAM. The storage device may be, for example, a register, a magnetic storage device such as a hard disk device, an optical storage device such as an optical disk device, an internal or external hard drive, a server, a solid-state storage device, CD-ROM, DVD, other optical or magnetic disk storage, or other storage devices. Computer-executable instructions include, for example, instructions and data which cause the processor system 10 to perform a certain function or group of functions. When the instructions are executed by the processor 12, the functions of each unit of the system and the like are implemented. The instructions may be a set of instructions constituting a program or an instruction for causing an operation on the hardware circuit of the processor.

Data, including subject voice data, subject non-voice data, and other data, such as reference voice data, reference abnormal voice data, and standard voice data may be stored in a database in the storage 13, such as the memory or another storage device. Such data may also be provided to the processor 12 by an input device 16, such as a keyboard, touchscreen, mouse, data acquisition device, network device, or any other suitable input device. Exemplary data acquisition devices may include an imaging system or device, such as an endoscope, a subject monitor, or any other suitable system or device capable of collecting or receiving data regarding the subject. Subject data may include voice data and/or non-voice data, and may include any of static data, dynamic data, and longitudinal data. In one embodiment, subject voice data collected by an endoscope are provided to the processor to diagnose a severity of a condition. In one embodiment, data, such as subject, standard, and reference voice data, as well as non-voice data may be stored in a database or various databases accessible by the processor 12.

The various components of the diagnostic system 10 and the like may be connected with each other via any types of digital data communication such as a communication network 22. Data may also be provided to the processor system 10 through a network device 20, such as a wired or wireless Ethernet card, a wireless network adapter, or any other devices designed to facilitate communication with other devices through a network 22. The network 22 may be, for example, a Local Area Network (LAN), WAN (Wide Area Network), and computers and networks which form the Internet. The system 10 may exchange data and communicate with other systems through the network 22. Although the system shown in FIG. 1 is shown as being connected to a network, the system may be also be configured to work offline.

Results, including diagnoses of conditions causing the abnormal or altered voice, output by the processor 12 may be stored in accordance with one or more algorithms in the memory 14, may undergo additional processing, or may be provided to an operator via an output device 18, such as a display and/or a printer. Based on the displayed or printed output, an operator may request additional or alternative processing or provide additional or alternative data, for example, via an input device 16.

FIG. 2 shows an exemplary method for diagnosing a subject as having a particular condition based on abnormal voice data. Conditions may include diseases such as the cold or flu, or conditions that cause growths to form on the vocal cords such as thyroid cancer. Other conditions can include esophageal cancer and throat cancer. As shown in FIG. 2, a subject voice data 30 of a subject is obtained. The subject voice data 30 is compared with a standard voice data 32 to generate a subject abnormal voice data 34, which is indicative of differences between the subject voice data and the standard voice data. The subject voice data 30 may be audio data collected by a recording device or a mobile device or any device that includes a speaker and a processor configured to record audio data. The subject voice data 30 can be collected when the user initiates a recording. Alternatively, the recording or mobile device can continuously collect the subject voice data when the user's voice is detected. The subject voice data 30 may be processed to improve contrast and/or extract or enhance features. The standard voice data is data of a person that is in a healthy state. The standard voice data may be an earlier recording of the subject's voice in a healthy state or may be the voice data of a different person in a healthy state. As discussed in more detail below, the different person may be selected based on one or more shared characteristics with the subject, such as age, race, and sex.

The subject abnormal voice data 34 is then compared 40 with the reference abnormal voice data 38. All of the voice data may be standardized into one or more common or similar formats to facilitate analysis and comparison. The subject is diagnosed 44 as having a particular condition based at least on part on the comparison between the subject abnormal voice data and the reference abnormal voice data. The diagnosis 44 may also be made by taking other data 42 and analysis into consideration, including non-voice data, such as clinical data, laboratory data, subject history, family history, subject vital signs, results of various tests (e.g., genetic tests), and any other relevant non-voice data. Based on the subject and reference data, numerous reference and subject abnormal non-voice and voice data may be created. Then, a report 46 of the diagnosis 44 is output to an output device 18 (shown in FIG. 1), such as a display or a printer, or may be output to a database for storage, or to a user in a human-readable format.

The various voice data and data described herein may be stored in one or more databases to facilitate subsequent data analysis. Moreover, any or all of the foregoing comparisons may be performed either automatically by a data processing system, such as system 10, or by a medical professional, such as a doctor, or by some combination thereof, to facilitate automatic or manual diagnosis of the subject in step 44.

The abnormal voice data described herein may be generated through any suitable technique. For example, abnormal voice data can be a difference determined between two or more sets of voice data. For example, a subject abnormal voice data may be created by comparing standard voice data with the subject voice data. Likewise, reference abnormal voice data may be generated by comparing the standard voice data with each of the reference voice data. Abnormal voice data may be generated from voice data and/or one or more of numerical data, text data, waveform data, voice data, video data, and the like.

In another aspect, reference data, including voice data and non-voice data, may be collected from people or groups of people. Such people may include healthy people that are not suffering a condition that causes an abnormal voice, and other people suffering from various conditions causing an abnormal voice and differing severity levels thereof, including, for example, cold, flu, growth on vocal cords, thyroid cancer, esophageal cancer, and throat cancer. The reference voice data and non-voice data may be standardized and categorized according to one or more characteristics. For example, such reference data may be categorized based on population characteristics, such as race, gender, or age of the people from which the data was collected. Standardized data permits average vocal characteristics to be calculated for healthy subjects and subjects with different severity levels of a particular condition.

An exemplary method for generating abnormal voice data, indicative of differences between a subject's voice and a reference voice, is illustrated in FIG. 3. In this embodiment, reference voice data 50 is categorized and standardized in step 52. Reference voice data and non-voice data may be collected from people and categorized or standardized according to one or more desired characteristics, such as age, gender, or race. While the presently illustrated embodiment is described with respect to voice data, it is noted that reference non-voice data and subject non-voice data may also, or instead, be used to generate the abnormal voice data discussed herein.

The method may include a step 54 of selecting a subset of the reference voice data based on a subject characteristic. For instance, if a subject is a thirty-five year old Japanese man, a subset of the reference voice data grouped to include reference voices pertaining to men between thirty and forty years of age may be more relevant for comparative purposes than a group of reference voice data composed of data collected from men between sixty and seventy years of age. Similarly, a subset of the reference voice data group to include reference voices pertaining to Japanese men may be more relevant for comparative purposes that a group of reference voice data composed of data collected from Caucasian men. A subset of reference voice data collected from Japanese men between thirty and forty years of age may be the most relevant for comparative purposes.

Once a desired group of reference voice data is selected, the matched reference voice data 56 may be compared to voice data 60 of the subject in step 58. In other embodiments, non-voice data of the subject may instead or also be compared to match reference non-voice data, as described above. Additionally, the various data may be processed and categorized in any suitable manner to facilitate such comparisons.

Additionally, reference data may be categorized and sorted into standardized databases, such as through an exemplary method shown in FIG. 4. The method may include acquiring reference data 70, which may include voice and non-voice data from various people, and categorizing the data in step 72. For example, the reference data 70 may be categorized into various groups, such as normal (healthy) subject data 74, data 76 of subjects clinically diagnosed with a first condition, such as cold/flu, data 78 of subjects diagnosed with a second condition, such as having one or multiple growths on the vocal cords, and data 80 of subjects diagnosed with a third condition, such as thyroid cancer. The data 74, 76, 78, and 80 may be stored in respective databases 82, 84, 86, and 88. Such databases may be stored on a server, in one or more memory devices, and/or in other suitable media. The data 76 of subjects diagnosed with cold/flu, the data 78 of subjects diagnosed with one or multiple growths on the vocal cords, and the data 80 of subjects diagnosed with thyroid cancer may further be categorized and/or divided into different databases based on severity level, as discussed in more detail below. Such databases may be continuously or periodically updated as more subjects are diagnosed.

Based on the subject and reference voice data and non-voice data discussed above, numerous reference and subject abnormal data and voice data may be created. By way of example, an exemplary method 100 for generating and analyzing such abnormal data is shown in FIG. 5. The method 100 includes acquiring reference data for: normal subjects without any diagnosed conditions that cause an abnormal or altered voice (data 102), subjects clinically diagnosed with the cold or flu (data 104), subjects diagnosed with esophageal cancer (data 105), subjects diagnosed with a growth on the vocal cord (data 106), subjects diagnosed with throat cancer (data 107), and subjects diagnosed with thyroid cancer (data 108). The method 100 may also include acquiring subject data 110. The method 100 may acquire reference voice data for other disorders that affect a person's voice, which may be processed in a manner similar to those discussed in the present example. Indeed, the present processing techniques may also be applied to other disorders unrelated to conditions that affect a person's voice.

Calculating Patient Subject Scores for Diagnosing Severity

In step 112, the standard data 102 may be compared to each of the other data 104, 105, 106, 107, 108, and 110, to generate cold/flu deviation data 116, esophageal cancer deviation data 117, growth on vocal cord deviation data 118, throat cancer deviation data 119, thyroid cancer deviation data 120, and subject deviation data 114, all of which may represent deviations from the standard/normal data 102. Such deviation data may compare specific characteristics of voice data between the subject data and: (i) the reference data for the particular condition, and (ii) the normal reference data. For example, characteristics of voice data may include phonation, loudness, rate, basic frequency data to determine pitch, and spectrum envelope data. Spectral envelope data can be useful in sound analysis because of its ability to capture important properties of sound such as voice quality and any blurring of the voice, which are typically indicative of a pharyngeal disease. This data is calculated using frequency and log magnitude by determining the Fourier transform and the discrete cosine transform. The Fourier transform of collected voice data (measured in Hertz) can be used to calculate the log magnitude (measured in decibels). From this, discrete cosine transform can also be obtained. This process is illustrated in FIG. 12A-C. FIGS. 12A-C shows an exemplary method for determining vocal characteristics such as spectral envelope data. Specifically, FIG. 12A shows audio data obtained from the subject. FIG. 12B shows a complex spectrum of the log magnitude obtained by the Fourier transform of the audio data shown in FIG. 12A. FIG. 12C is spectral envelope data obtained by the discrete cosine transform of the complex spectrum shown in FIG. 12B.

The spectral envelope data obtained in FIG. 12C is used to determine the rate of change for the subject and a normal rate of change. The rate of change information for the subject is then used to determine whether the voice data is abnormal or normal.

The rate of change information is determined as follows:

First, a subject spectral envelope and a normal spectral envelope are created based on data shown in FIGS. 12A-C. Next, the spectral envelope is divided into 150 regions as shown in FIG. 13. For example, S_{A-C} as shown in FIG. 13 are each divided into 50 regions to define a total of 150 regions. The rate of change is determined at each point of the spectral envelope. The formula for determining the rate of change is as follows: (logₙₒᵣₘₐₗ−log_{subject})/logₙₒᵣₘₐₗ×100. This formula provides the rate of change at the specific point identified on the spectrum envelope.

For example, referring to FIG. 13, at Q₁ on the normal spectral envelope, where the normalized frequency is 4 radians, the log magnitude is 155 dB. At Q3 on the subject's spectral envelope, where the normalized frequency is also 4 radians, the log magnitude is 120 dB. The rate of change between normal values and the subject's values at 4 radians is calculated as follows:

(155 dB−120 dB)/155 dB×100=22.58%

Referring to FIG. 13, Q₂ on the normal spectral envelope, where the normalized frequency is 140 radians, the log magnitude is 18 dB. At Q₃ on the subject's spectral envelope, where the normalized frequency is also 140 radians, the log magnitude is 16 dB. The rate of change between normal values and the subject's values at 140 radians is calculated as follows: (18 dB−16 dB)/18 dB×100=11.11%

In order to determine whether voice data is abnormal, a score is calculated and compared with the values in the table shown in FIG. 9, discussed in more detail below. In general, if the rate of change is less than 20%, the subject's voice data can be judged as being normal. If the rate of change is 20% or more, it can be judged as being abnormal. This rate of change also indicates the level of hoarseness in a subject's voice.

To calculate the score for a subject, a section value is multiplied by the value corresponding to the rate of change. Section values are determined as follows: For the values in section S_{A}, all calculated rates of change are multiplied by 0.8. For the values in section S_{B}, all calculated rates of change are multiplied by 0.5. For the values in section S_{C}, all calculated rates of change are multiplied by 0.1. Values for the rate of change are determined as follows: 0.1 if the rate of change is less than 10%, 0.5 if the rate of change is 10% or more and less than 20%, and 0.8 if the rate of change is 20% or more.

For example, for 4 radians, which is in section S_{A}, the calculated rate of change is 22.58%. The section value for section S_{A} is 0.8. The value for the rate of change is 0.8 because the calculated rate of change is 20% or more. Therefore, the score for 4 radians is calculated as follows: 0.8×0.8=0.64

At 140 radians, which is in section S_{C}, the calculated rate of change is 11.11%. The section value for Section S_{C} is 0.1. The value for the rate of change is 0.5 because the calculated rate of change is 10% or more and less than 20%. Therefore, the score for 140 radians is calculated as follows: 0.1×0.5=0.05.

The score at each of the 150 points are calculated and then added together. The sum is the score compared to the values referenced in FIG. 9 to determine whether the voice data is abnormal. This manner of determining whether the voice data is abnormal can be done with improved accuracy. It can also be done with the convenience of using the subject's own device. Because this scoring can be done with a subject's own device, this allows the subject to frequently monitor his condition without frequent visits to a health care professional and allow for early detection of any serious conditions. Additionally, the data is all stored on the subject's device, which allows the subject to provide the data to his health care professional if necessary. The stored data and record keeping may also track location information to provide contextual information for future reference by the subject and a health care professional. Further, the ability for a subject to monitor his condition provides a low-cost option for long-term monitoring of a condition for those who may find seeking help from a health care professional too expensive or may not have access to a healthcare professional.

In step 122, such abnormal data may be analyzed. For example, a subject abnormal voice data or data may be compared to representative reference abnormal voice data or data for each of the above noted conditions to facilitate diagnosis of the subject with respect to one or more of such conditions. Additionally, reference clinical data 124, subject clinical data 126, and other data 128 may also be analyzed by a data processing system or a user to facilitate diagnosis. In one embodiment, such analysis may include pattern matching of subject voice data and reference voice data, and confidence levels of such matching may be provided to a user. Finally, results 130 of the analysis may be output to storage or to a user via, for example, an output device 18, such as a display or printer. For example, a screen of a mobile device, such as a smart phone, can display the following:

“I detected an abnormality in your voice. Do you have a cold?”

“Recommended: Go to the hospital.”

“Condition Severity: Low Risk”

“Condition Severity: Moderate Risk”

“Condition Severity: High Risk”

“Abnormal Condition: Hoarseness”

“Normal Condition”

A method 130 for analyzing the data discussed above and diagnosing a subject is illustrated in FIG. 6. In step 132, one or more subject abnormal voice data, which may include characteristics of voice data as discussed above (for example, phonation, pitch, loudness, rate, basic frequency data, spectrum envelope data, and aperiodic component data to detect hoarseness), may be compared to one or more reference abnormal voice data, such as those previously described. Notably, the reference abnormal voice data may include abnormal voice data representative of one or more conditions, as well as various severity levels of the one or more conditions.

Based on such comparisons, one or more subject conditions and/or severity levels may be identified in step 134 and diagnosed in step 138. In some embodiments, such as a fully automated embodiment, steps 134 and 136 may be combined. In other embodiments, however, the identification and diagnosis may be performed as separate steps. For instance, the data processing system 10 may identify various potential conditions or severity levels and present the identified conditions or severity levels to a user for diagnosis. A report 138 may include an indication of the identified subject condition(s) or severity levels, the diagnosis, or both.

The extent of subject deviation from standardized data may also be translated into one or more abnormal scores, which may, in one embodiment, be generated through the methods shown in FIGS. 7 and 8. An exemplary method 140 of FIG. 7 may include accessing subject voice data 142 and reference voice data 144, including standard voice data and voice data representative of a particular condition and/or severity level thereof. Such voice data may be received from any suitable source, such as a database or an imaging system, such as an endoscope. The voice data 142 and 144 may include voice data collected from a wide range of sources. The reference voice data 144 may be standardized according to any desired characteristics. For instance, in one embodiment, the reference voice data 144 may generally represent features of normal individuals with certain characteristics, for example, characteristics similar to the subject. In step 146, the subject voice data 142 and the reference voice data 144 may be compared to determine deviations of the subject voice data 142 from the reference voice data 144. In one embodiment, such differences may generally represent deviation, for example, structural differences between the subject and normal (e.g., healthy) subjects.

The method 140 may also include calculating 148 one or more subject voice data abnormal scores for differences between the subject voice data 142 and the reference voice data 144. Such abnormal scores may be indicative of an array of structural deviations of the subject relevant to the reference voice data. The subject voice data abnormal scores may be calculated in various manners, such as based on projection deviation, single pixel (2D) deviation, single voxel (3D) deviation, or on any other suitable technique. The calculated subject voice data abnormal scores 150 may then be stored in a database 152, output to a user, or may undergo additional processing in one or more further steps 154.

Specific parameters obtained from the subject abnormal voice data are compared with specific parameters obtained from the subject normal voice and/or the reference normal voice. As discussed above, the vocal parameters include phonation, pitch, loudness, rate, basic frequency data, spectrum envelope data, and aperiodic component data to detect hoarseness.

A detailed discussion of the scoring system the processor can use to diagnose the severity of a subject's condition is discussed in detail below.

For example, as shown in FIG. 9, in 5000 patients who have a score of 0-20, 3000 of them or 60% of them could be normal; 1500 of them or 30% of them could have a low degree condition; 450 or 9% of them could have a middle degree condition; and 50 or 1% of them could have a high degree condition. In 3000 patients who score 20-40, 2000 or 67% of them could be normal, 500 or 17% of them could have a low degree condition; 400 or 13% of them could have a middle degree condition; and 100 or 3% of them could have a high degree condition. In 2000 patients who score 40-60, 200 or 10% of them could have a normal condition; 400 or 20% of them could have a low degree severity of the condition; 600 or 30% of them could have a middle severity of the degree condition; and 800 or 40% of them could have a high degree severity of the condition. An example of a low degree severity of the condition is a cold. An example of a middle degree condition is vocal cord polyps. An example of a high degree condition is cancer. Cancers could include esophageal cancer, pharyngeal cancer and thyroid cancer.

From these comparisons, it is possible to determine the probability that a subject has a particular condition. For example, if the subject has a score of 30 points, the probability of the subject has a cold is 17%, the probability the subject has a growth on his vocal cords is 13%, the probability the subject has cancer is 3%.

FIG. 8 shows an exemplary method 160 for calculating non-voice data abnormal scores. The method 160 may include accessing subject non-voice data 162 and reference non-voice data 164. The non-voice data may be received from any suitable source, such as a database, a computer, or subject monitor. The subject non-voice data 162 may include any non-voice data information collected for the purpose of diagnosing the subject, such as clinical data, laboratory data, subject history, family history, subject vital signs, and the like, and may also include results of other tests, such as genetic tests and so forth. The reference non-voice data 164 may include similar data, which may be standardized based on one or more characteristics of the persons from whom it was obtained. In one embodiment, the subject non-voice data 162 and reference non-voice data 164 may include one or both of numeric data and enumerated data, each of which may be continuous or discrete. The reference non-voice data 164 may be data representative of features of normal persons with particular characteristics, such as those similar to the subject. In step 166, the subject non-voice data 162 may be compared to the reference non-voice data 164 to identify differences between the data. In one embodiment, such differences may generally represent a deviation, such a structural deviation, of the subject from normal (e.g., healthy) individuals.

Additionally, the method 160 may include a step 168 of calculating one or more subject non-voice data abnormal scores for differences between the subject non-voice data 162 and the reference non-voice data 164. Various techniques may be used to calculate the subject non-voice data abnormal scores, including, for example, z-score deviation or distribution analysis. Of course, it will be appreciated that other calculation techniques may also or instead be employed in other embodiments. The calculated subject non-voice data abnormal scores 170 may be stored in a database 172, output to a user, or may undergo additional processing in one or more further steps 174.

A processor can be configured to compare differences between subject voice data and reference data to determine difference data. For example, in FIG. 11, the processor, by implementing a deep learning process described in detail below, generates normal voice data. This process is explained in more detail in the next section. The subject voice data and normal voice data are then compared and difference data is generated. This difference data is compared model difference data to determine a rate of change in order to determine if the severity of the subject's condition is low, moderate or high.

For example, in FIG. 11, the generated normal voice data is compared with the subject voice data for three patients, Patients A, B, and C. For Patient A, the subject voice data is indicated with data line B and normal voice data is indicated with data line A. The difference data is generated and compared to determine a rate of change for diagnosing the severity of Patient A's condition. For Patient B, the subject voice data is indicated with data line D and normal voice data is indicated with data line C. The difference data is generated and compared to determine a rate of change for diagnosing the severity of Patient B's condition. For Patient C, the subject voice data is indicated with data line F and normal voice data is indicated with data line E. The difference data is generated and compared to determine a rate of change for diagnosing the severity of Patient C's condition. Each data line compares the log magnitude to each normalized frequency. As seen in FIG. 11, a difference area between the subject voice data and normal voice data is small for Patient A, moderate for Patient B, and large for Patient C. This suggests that Patient A's condition is the least severe and Patient C's condition is the most severe. The rate of change in the difference data for each of Patients A, B, and C are obtained by comparing the log magnitude for each normalized frequency in FIG. 11. Specifically, the rate of change is obtained by comparing data line A with data line B, data line C with data line D, data line E with data line F. The rate of change is determined as discussed above with respect to FIG. 13 using the following formula: (SEₙₒᵣₘₐₗ−SE_{subject}) SEₙₒᵣₘₐₗ×100. Scoring is then calculated to determine if the severity of the subject's condition is low, moderate or high.

This scoring system can be applied when the reference abnormal voice data is compared with the reference normal voice data. The scoring system can also be applied when the subject abnormal voice data is compared with the reference abnormal voice data.

This scoring method is applied to all known parameters of the subject abnormal voice data. After obtaining scores for each parameter, a total score is determined. For example, a total score can range between 0-60. When a total score is obtained, the total score is compared to scores obtained by other patients. For example, there could be 10,000 patients with a score of 0-20 and 5,000 of them have a cold, 1,000 of them have a growth on the vocal cord, 100 of them have throat cancer and 50 of them have esophageal cancer. There could also be 2,000 patients with a score of 20-40 where 800 of them have a cold, 500 of them have a growth on the vocal cord, 50 of them have throat cancer and 10 of them have esophageal cancer.

Technical effects of the present disclosure include the accurate and consistent diagnoses of various conditions and severity levels thereof, as well as providing decision support tools for user-diagnosis of subjects. For example, technical effects may include the visualization of subject voice data and non-voice data information together in a holistic, intuitive, and uniform manner, facilitating accurate and objective diagnosis by a user. Additionally, the present systems, methods, and computer-readable media enable the generation of subject abnormal voice data and reference abnormal voice data of known conditions and/or severity levels thereof, and the combination of such voice data with other clinical tests, to facilitate quantitative assessment and diagnosis of conditions and their severity level. The disclosed systems, methods, and computer-readable media enable analysis of multiple parameters, including both voice data and non-voice data, to accurately and objectively diagnose severity levels of conditions.

In some embodiments, a system may be programmed or otherwise configured to gather clinical information and create integrated comprehensive views of the progression of statistical deviations of data of an individual subject from one or more normal subject populations over time from longitudinal data. In other words, subject voice data and/or non-voice data at a particular point in time may be compared to subject voice data and/or non-voice data collected at an earlier point in time to determine a change in the data of the subject over time. The change in the subject data over time may be used to facilitate diagnosis, for example, diagnosis of cancer and/or a severity thereof. In addition, the present systems, methods, and computer-readable media provide structured integrated comprehensive views of the deviation of the clinical information across a given diseased subject population when compared against a population of normal controls, both at a single point in time and across multiple time points (longitudinally). Such comprehensive views described herein may display a normative comparison to thousands of standardized and normalized data values concurrently. The resulting comprehensive view can provide patterns of deviations from normal that may indicate a characteristic pattern corresponding to known conditions or abnormalities and severity levels thereof.

Using the presently disclosed techniques, a user may be able to easily compare the results of one parameter with another, and draw conclusions therefrom. To facilitate such analysis, the various parameters may be standardized and normalized. Further, in some embodiments, an integrated comprehensive view of clinical data of a specific population of people with respect to a population of normal subjects is provided. The view may include disparate types of clinical data, including both voice data and non-voice data in a manner that makes it easy for humans to distinguish the distribution of clinical parameter results across condition populations. Although various graphs can be used to analyze results for a single clinical parameter across populations, they are quite cumbersome and impractical when it comes to visualizing and analyzing a larger number of parameters. The present disclosure analyzes multiple parameters, including both voice data and non-voice data to accurately and objectively diagnose severity levels of conditions.

The subject's information is input into the processor 12 including the subject's age, sex, race, country of origin, residence information. It is also possible to include the subject's family members' information.

Employing Machine Learning

Some embodiments may employ systems and methods for voice data analytics using machine learning. For example, the narrow band voice data, including difference voice data, the diagnosis (e.g., cancer severity and/or cancer stage), and medical record information (e.g., information of patient's age, gender, nationality, medical history, and other tests, such as X-ray imaging examination, CT examination, MRI examination, PET examination, ultrasound examination, pathological examination results, or the like) may be input into a system, such as the system 10 (FIG. 1). The system may be configured to execute a program to “learn” the voice data and the cancer diagnosis, and determine a relationship between the voice data and the cancer. For example, the program may be taught to analyze voice data by providing the program with training information that includes previously-analyzed voice data and associated diagnoses, as well as other relevant clinical, demographic, and external data. The system may learn the voice data and the diagnosis (e.g., cancer severity and/or cancer stage), and determine a relationship between the voice data and the diagnosis (e.g., cancer severity and/or cancer stage). The system may also learn the voice data, the cancer, the stage and/or severity of cancer, and the medical record information to determine each relationship.

The computer program may be configured to perform machine learning using various types of methods and mechanisms. For example, the computer program may perform machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using any or all of these approaches, a computer program may ingest, parse, and understand data and progressively refine relationships and models for data analytics.

The processor can undergo deep learning in which the processor learns a large volume of voice data about a subject's normal condition. Deep learning is a machine learning technique using multiple data processing layers to recognize various structures in data sets and accurately classify the data sets. For instance, a deep learning model may be trained to generate corresponding normal voice data from abnormal voice data, such as reference voice data indicative of a particular level of severity or an abnormal voice data. The model may be trained based on normal training voice data only. Such normal training voice data may be voice data of healthy subjects that are substantially free of abnormalities. The model may be trained via unsupervised learning in which the model extracts and learns features and patterns of the normal training voice data. That is, the model may analyze raw normal voice data to identify features and patterns of normal voice data without external identification.

Once learned, the model can receive voice data from a subject to predict whether it is normal or abnormal voice data. For example, a subject's input voice data is obtained and compressed with an encoder. Using a decoder, the input voice data that is normal is restored, and voice data that is abnormal is not restored. The processor can learn from daily conversations the subject has and this voice data can be downloaded to the processor or stored on a cloud based storage. Deep learning techniques can include using the variational auto-encoder model.

When abnormal voice data belonging to the subject is input into the processor's learning model, the log magnitude of the abnormal data is corrected to a normal value without changing the value of the normalized frequency of the data. Subsequently, the processor is configured to generate graphs that include the log magnitude of the abnormal data with normalized frequency values. As shown in FIG. 10, voice data for Patients A, B and C are obtained. In the case of Patient A, the obtained voice data is indicated with data line b. Using deep learning, the processor is configured to determine a normal data line a. The processor then compares data lines a and b to make a diagnosis on severity. In the case of Patient A, the severity is low. In the case of Patient B, the obtained voice data is indicated with data line c. Using deep learning, the processor is configured to determine a normal data line d. The processor then compares data lines c and d to make a diagnosis on severity. In the case of Patient B, the severity is moderate. In the case of Patient C, the obtained voice data is indicated with data line e. Using deep learning, the processor is configured to determine a normal data line f. The processor then compares data lines e and f to make a diagnosis on severity. In the case of Patient B, the severity is moderate. The process of diagnosing the severity of a patient is described in further detail

The system then performs data analytics to determine meaningful patterns in voice data and non-voice data and builds models based on these determined patterns, which can be used to automatically analyze voice data and other medical data. In some embodiments, after developing a model using training information, the system may update the model based on feedback designating a correctness of the training information or a portion thereof. For example, the system may update a model based on clinical results associated with one or more voice data included in the training information. In other embodiments, a user may manually indicate whether diagnostic information included in the training information was correct as compared to an additional (e.g., later established diagnosis).

After the model is developed, the system may receive voice data and/or non-voice data for analysis and may diagnose the subject as having cancer and/or a severity or stage thereof based on the foregoing information. For example, the subject's narrow band voice data and medical record information may be input into the system. The system can diagnose a condition based on learned information and the narrow band voice data using the model. The system may identify the cancer stage and/or severity based on the above diagnosis and medical record information (e.g., presence or absence of cancer metastasis). The system may output to a user a diagnosis of the presence or absence of cancer and the stage and/or severity of cancer. As more and more subjects are diagnosed with various severity levels and stages of cancer, the system may update its models accordingly.

Technical effects of the present disclosure include the accurate and consistent diagnoses of various conditions and severity levels thereof, as well as providing decision support tools for user-diagnosis of subjects. For example, technical effects may include the visualization of subject voice data and non-voice data information together in a holistic, intuitive, and uniform manner, facilitating accurate and objective diagnosis by a user. Additionally, the present systems, methods, and computer-readable media enable the generation of subject abnormal voice data and reference abnormal voice data of known conditions and/or severity levels thereof, and the combination of such voice data with other clinical tests, to facilitate quantitative assessment and diagnosis of conditions and their severity level. The disclosed systems, methods, and computer-readable media enable analysis of multiple parameters, including both voice data and non-voice data, to accurately and objectively diagnose severity levels of conditions.

There are four major treatments for esophageal cancer: endoscopic resection, surgery, radiation therapy and drug therapy (for example chemotherapy). A subject can be treated with one or more of these treatments.

For early stage throat cancer, treatment can be performed by radiation or surgery. Surgery can include preserving the larynx (laryngeal preservation surgery). Treatments can also include a combination of radiation therapy and surgery depending on the subject's condition. For later stage throat cancer, a laryngectomy can be performed wherein the entire larynx is removed. Treatments are also available that include radiation and drug therapy, including chemotherapy, to help preserve the larynx and subject's voice.

Thyroid cancer can be treated with surgery, radiation therapy, drug therapy, including endocrine therapy, hormone therapy, molecular targeted therapy and chemotherapy. Treatment typically requires surgery except in cases of high grade undifferentiated cancer. Undifferentiated cancer is a rare cancer found in about 1% of thyroid cancers. The nature of papillary cancer and follicular cancer that has already existed for many years can suddenly and turns into an undifferentiated cancer.

For treatment of a vocal cord polyp, it is important to avoid speaking. If additional treatment is required, anti-inflammatory analgesics or inhaled steroids can be used. These drugs usually cause any inflammation to subside over several months. However, if inflammation does not reduce, surgery can be performed under general anesthesia. A device can be used to remove and extract the polyp.

It will be appreciated that any of the above-disclosed and other features and functions, or alternatives thereof, may be desirably combined into many other different systems or applications. Also, various presently unforeseen or unanticipated alternatives, modifications, variations, or improvements therein may be subsequently made by those skilled in the art, and are also intended to be encompassed by the following claims.

What is claimed is:

1. A system for determining a condition affecting voice, comprising:
a processor programmed to:
obtain voice data of the subject;
compare the voice data with reference voice data;
determine a rate of change between the voice data of the subject and the reference voice data; and
obtain a score for the subject based on the rate of change in order to determine whether the voice data of the subject is abnormal.

2. The system according to claim 1, wherein the score is obtained by dividing a spectral envelope into a plurality of regions.

3. The system according to claim 2, wherein each region is provided in one of a plurality of sections.

4. The system according to claim 1, wherein the reference voice data is voice data of a healthy individual.

5. The system according to claim 2, wherein the processor is further programmed to assign predetermined values for each of the plurality of equally divided sections.

6. A method for determining a condition affecting voice, comprising:
obtaining voice data of a subject;
comparing the voice data with reference voice data;
determining a rate of change between the voice data of the subject and the reference voice data;
obtaining a score for the subject based on the rate of change in order to determine whether the voice data of the subject is abnormal; and
determining whether the subject has a specific condition the obtained score.

7. A computer-readable storage medium storing a computer-executable programmed to perform functions comprising:
obtaining voice data of a subject;
comparing the voice data with reference voice data;
determining a rate of change between the voice data of the subject and the reference voice data;
obtaining a score for the subject based on the rate of change in order to determine whether the voice data of the subject is abnormal; and
determining whether the subject has a specific condition the obtained score.

8. The system for determining a condition affecting voice according to claim 1, wherein the processor is configured to update the reference voice data based on normal voice data obtained from the subject and clinical results data.

9. The system for determining a condition affecting voice according to claim 1, further comprising a display configured to display diagnosis information based on the score obtained by the processor.

10. The system for determining a condition affecting voice according to claim 1, further comprising a display configured to display instructions based on the score obtained by the processor.
